(19) United States
(12) Patent Application Publication
(10) Pub. No.: US 2024/0277003 A1
(43) Pub. Date: Aug. 22, 2024

(54) CHEWABLE GEL COMPOSITIONS WITH EXTENDED ORAL RESIDENT TIME AND METHODS OF MAKING AND USING THEREOF

(71) Applicant: Seattle Gummy Company, Renton, WA (US)

(72) Inventors: Feng WAN, Sammamish, WA (US); William Brenden Carlson, Shoreline, WA (US)

(73) Assignee: Seattle Gummy Company, Renton, WA (US)

(21) Appl. No.: 18/571,254

(22) PCT Filed: Jun. 19, 2022
(86) PCT No.: PCT/US22/34127

§ 371 Date: Dec. 18, 2023

Related U.S. Application Data
(60) Provisional application No. 63/212,122, filed on Jun. 18, 2021.

(51) Int. Cl. A23G 3/42; A23G 3/36; A23G 3/38; A23G 3/44; A61K 9/68; A61K 31/135; A61K 31/198; A61K 31/4545; A61K 31/495; A61K 31/522; A61K 36/258; A61K 36/287; A61K 36/355; A61K 36/9068

(52) U.S. Cl. CPC A23G 3/42; A23G 3/364; A23G 3/38; A23G 3/44; A61K 9/0058; A61K 31/135; A61K 31/198; A61K 31/4545; A61K 31/495; A61K 31/522; A61K 36/258; A61K 36/287; A61K 36/355; A61K 36/9068

(57) ABSTRACT

A gummy composition, comprising a semi-solid chewable base comprising a gum composition, a gelling composition, and a bonding composition, wherein the gelling composition comprises a protein-based gelling agent, a carbohydrate-based gelling agent, or a combination thereof, wherein the gum composition and the gelling composition have a ratio of not more than 4:1 w/w, and wherein the bonding composition comprises a mono-, di- tri-, or oligosaccharide, a sugar alcohol, or a combination thereof.

### CROSS-REFERENCES TO RELATED APPLICATIONS

This application claims the benefit of priority from, and hereby incorporates by reference the entire disclosure, co-pending U.S. Provisional Applications for Patent Ser. No. 63/212,122, filed Jun. 18, 2021.

### TECHNICAL FIELD

This application relates to gummy composition having an extended oral residence time and slowly dissolve when chewed.

### BACKGROUND

Unless otherwise indicated herein, the materials described in this section are not prior art to the claims in this application and are not admitted being prior art by inclusion in this section.

Pharmaceuticals and dietary supplements are available in a variety of dosage forms including tablets, capsules, soft-gels, powders, chewable tablets, and liquid suspensions. Tablets, capsules and soft gels are difficult for individuals who have difficulties swallowing pills; powders are often difficult to administrate; and the dosing with liquid dosage forms is not precise, which can lead to the administration of inaccurate dose.

The chewy texture of a gummy distinguishes it from other confectionaries. Comparing to pills and capsules, gummies are convenient, easy to swallow, and more appetizing therefore reaching far more customers than traditional dosage forms.

One of the advantages of using gummy to deliver the actives is that chewing gummy provides opportunity for prolonged contact of the actives with the oral mucosal membrane and within the oral cavity, which could facilitate, on the one hand, oral mucosal absorption, and on the other hand, oral health treatment with the bioactive ingredients (i.e., bioactives).

Traditional gummy has very short oral resident time before it dissolves through chewing. There remains a need for a gummy formulation that can provide a prolonged/extended oral resident time within the oral cavity therefore maximizing the mucosal absorption of the bioactives, or the exposure of oral cavities to the bioactives.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid chewable composition (i.e., gummy composition) having extended oral resident time when chewed. In one embodiment, the gummy composition comprises a semi-solid chewable gel base (i.e., gummy base) comprising a gum composition, a gelling composition, and a bonding composition.

The gum composition may include a gum. Gums may be a polysaccharide, a glycoprotein, or a mixture thereof; it may be natural or synthetic. In one embodiment, the gum composition comprises a plant gum, a glycoprotein, or a combination thereof. Example plant gum includes xantban gum, guar gum, locust bean gum, gum Karaya, gum Tragacanth, Tara gum, gum Arabic, or a combination thereof. In one embodiment, the gum composition comprises gum Arabic. In one embodiment, the gum composition comprises xanthan gum and locust bean gum.

The gelling composition may include a protein-based gelling agent, a carbohydrate-based gelling agent, or a combination thereof. Example protein-based gelling agent comprises gelatin, egg white protein, albumin, or a combination thereof. Example carbohydrate-based gelling agent may include starch, pectin, carrageenan, alginate, agar, gellan, konjac, a cellulose derivative, or a combination thereof. Example starch may be corn starch, tapioca starch, arrowroot starch, potato starch, wheat starch, yarn starch, mung bean starch, rice starch, bracken starch, sago, modified starch, pre-gelatinized starch, or a combination thereof. Example cellulose derivative may include a cellulose derivative. In one embodiment, the cellulose derivative comprises methylcellulose (MC), hydroxypropylmethyl cellulose (HPMC), hydroxyethyl methyl cellulose (HPMC), hydroxyethyl cellulose (HEC), carboxymethylcellulose (CMC), or a combination thereof.

In one embodiment, the gum composition comprises gum Arabic or xanthan gum and the gelling composition comprises gelatin. In one embodiment, the gum composition comprises xanthan gum, Arabic gum, guar gum, gum tragacanth, or a combination thereof and the gelling composition comprises agar, gellan, or a cellulose derivative. In one embodiment, the gum composition comprises xanthan gum and the gelling composition comprises agar, carboxymethyl cellulose, methyl cellulose, or a combination thereof.

The gum composition and the gelling composition have a ratio of not more than 4:1 w/w. In one embodiment, the gum composition and the gelling composition have a ratio from 4:1 w/w to 1:2 w/w. In one embodiment, the gum composition and the gelling composition have a ratio of not more than 3.5:1. In one embodiment, the gum composition and the gelling composition have a ratio of 3.9:1, 3.8:1, 3.7:1, 3.6:1, 3.5:1, 3.4:1, 3.3:1, 3.2:1, 3.1:1, 3:1, 2.9:1, 2.8:1, 2.7:1, 2.6:1, 2.5:1, 2.3:1, 2:1, 1.8:1, 1.5:1, 1.2:1, 1.1, 1:1.5, 1:3, 1:2, or 1:1 w/w.

In one embodiment, the gummy composition comprises the gum composition from 20% to 60% w/w, 25% to 40% w/w. In one embodiment, the gummy composition comprises the gelling composition from 0.5% to 15% w/w. In one embodiment, the gummy composition comprises the protein-based gelling agent from 5% to 15% w/w. In one embodiment, the gummy composition comprises the carbohydrate-based gelling agent from 0.5% to 5% w/w.

The bonding composition may include a mono-, di- tri-, or oligosaccharide, a sugar alcohol, or a combination thereof. Example mono-, di-, tri- or oligosaccharides include sucrose, glucose, fructose, L-fructose, L-glucose, L-galactose, psicose, palatinose, trehalose, tagatose, sorbose, D-maltose (1,4-diglucose), raffinose, or a combination thereof. In one embodiment, the bonding composition comprises essentially sucrose, glucose, fructose, or a combination thereof.

In one embodiment, the bonding composition comprises a low glycemic index (GI) sugar having a glycemic index value (GI) of less than 8, 10, 12, 15, 19, 20, 25, or 30. In one embodiment, the bonding composition comprises palatinose, trehalahose, tagatose, or a combination thereof. In one embodiment, the bonding composition comprises consists essentially of psicose, palatinose, and a third sugar selected from a group consisting of trehalose, sorbose, tagatose, or a combination thereof. In one embodiment, the bonding composition comprises essentially trehalose, palatinose, psicose, tagatose, sorbose, or a combination thereof.

In one embodiment, the bonding composition consists substantially of palatinose, trehalose, psicose, or a combination thereof. In one embodiment, the bonding composition consists substantially of tagatose, psicose, trehalose, or a combination thereof.

In one embodiment, the bonding composition comprises substantially sugar alcohol. Example sugar alcohol includes mannitol, maltitol, isomalt, erythritol, sorbitol, xylitol, or a combination thereof. In one embodiment, the bonding composition consists substantially of mannitol, maltitol, isomalt, or a combination thereof.

In one embodiment, the gummy composition comprises the bonding composition from 35% to 95%, 40% to 80%, 45% to 75%, 50% to 55%, 50% to 65%, 60% to 72%, or 65% to 85%.

The gummy composition may elastic or viscoelastic. In one embodiment, the gummy composition may be viscoelastic exhibiting both characteristics of a liquid and solid. In one embodiment, the gummy composition may have a water content from 2% to 20%, 8% to 12%, or 5% to 15% w/w. In one embodiment, the gummy composition immobilizes water molecules within it to form a rigid structure that is resistant to flow.

In one embodiment, the gummy compositions are configured to be chewed and slowly dissolved in oral cavity in not less than 1, 2, 3, 4, or 5 minutes, which is defined as “oral residence time.” In one embodiment, the oral residence time is not less than 30, 40, 50, 60, 70, 80, 90, or 120 seconds. In one embodiment, the oral residence time is from about 1 to 2 minutes, or about at least 2 minutes to 5 minutes. In one embodiment, the oral residence time is within 1 minute, less than 2 minutes, or in more than 3 minutes.

In one embodiment, the gummy composition has a dissolving time as tested in the disintegration apparatus from 35 mins to 75 mins, 40 mins to 60 mins, 50 mins to 90 mins.

In one embodiment, the gummy composition has a hardness of from 4 Kp to 20 Kp, 2 Kp to 15 Kp, 10 Kp to 20 Kp, 15 Kp to 20 Kp, 5 Kp to 15 Kp.

In one embodiment, the gummy composition has a springiness value from 0.3 to 1.0, 0.5 to 0.8, 0.6 to 0.9, 0.7 to 0.8. In one embodiment, the gummy composition has a cohesiveness value from 0.2 to 1.0.

In one embodiment, the gummy composition has a chewiness from 500 J to 20,000 J, 1000 J to 8000 J, 10,000 J to 20,000 J. In one embodiment, the gummy composition has a gumminess from 500N to 20,000N, 1000N to 8000N, 10,000N to 20,000N.

In one embodiment, the gummy composition has a pH of at least 4, 5, 6, 7, 8, or 9. In one embodiment, the gummy composition has a pH from 2.1 to 3.4, 3.2 to 3.9, 3.5 to 4.5, 3.9 to 5, 4.7 to 5.5, 5 to 6.3, or 7 to 9. In one embodiment, the gummy composition has a pH of at least about 6.

In one embodiment, the gummy composition has a glycemic index from 8 to 140, 8 to 15, 10 to 20, 15 to 29, 19 to 45, 38 to 70, 60 to 80, 70 to 120. In one embodiment, the gummy composition has a glycemic index of 0. In one embodiment, the gummy composition is substantially free of added sugar.

In one embodiment, the gummy composition is substantially free of sucrose, glucose, fructose, or a combination thereof. In one embodiment, the composition is substantially free of a sugar having a glycemic index of more than 35. In one embodiment, the composition is substantially free of sucrose, glucose, fructose, sugar alcohol, or any sugar substitute. Example sugar substitutes include sucralose, stevia, monk fruit extract, Splenda, etc.

The gummy composition may further comprise a bioactive composition to provide various health benefit. Heath benefit may systemic or local. Systemic health benefit may include promoting or supporting general health or treating, preventing or modulating diseases including, immune support, sleep aiding, anti-inflammatory, anti-anxiety, stimulating, cough suppressant, expectorant, anti-gas, fever or pain relief, anti-allergy, diuretic, anti-bacterial, anti-viral, deworming or anti-parasitic, analgesics, antiseptics, ant-ulcer, or a combination thereof. Local health benefit may include promoting oral health or treating oral diseases including anti-gingivitis, anti-caries, an anti-calculus, breath-freshen, or anti-bad breath.

In one embodiment, the bioactive composition comprises an herbal composition, an Active pharmaceutical Ingredient (API), an antioxidant composition, a vitamin composition, a mineral composition, an amino acid composition, a probiotics composition, or a prebiotics composition.

In one embodiment, the composition may promote oral health. Example actives include a vitamin, a mineral, an herbal active, a fluoride salt, a polishing agent, a tooth whitening agent, an anti-gingivitis agent, an anti-caries agent, an anti-calculus agent, an analgesic, an anti-inflammatory, an antiseptic, an anti-microbial agent, an anti-ulcer agent, an anti-bleeding agent, prebiotics, probiotics, a pH buffering agent, a chelating agent, a prebiotic, a probiotics, or a combination thereof.

The herbal composition may include Traditional Chinese Medicine (TCM) herbs. In one embodiment, the herbs may be promoting sleep including for example jujube seed, valerian root, or a combination thereof.

In one embodiment, the herbal composition may promote oral health, reducing swelling, relive pain, or a combination hereof. The gummy composition may provide oral health function such as anti-caries, reduce inflammation caused by gum disease, reducing gum bleeding, relieve toothache and gum pain, reduce bad breath, clean and refresh the oral cavity, or a combination thereof.

In one embodiment, the herbal composition comprises noroginseng, skullcap, isatisis root, corydalis, dandelion root, ginger, Zanthoxylum nitidum (Roxb.), Yan Husuo, Qingfengteng, Pueraria lobate, Sarcandra glabra (Thunb.) Nakai, Imperata cylindrica Beauv.var. major (Nees) C. E. Hubb., Ribes nigrum L. In one embodiment, the herbal composition comprises Dandelion, Isatisis (Radix Isatis), Scutellaria baicalensis, Radix Isatidis, or a combination thereof. In one embodiment, the herbal composition comprises Zanthoxylum nitidum (Roxb.), Yanhusuo, Guangui, or a combination thereof. In one embodiment, the herbal composition comprises Zanthoxylum nitidum (Roxb.), Centella asiatica, mentha, or a combination thereof. In one embodiment, the herbal composition comprises Zanthoxylum nitidum (Roxb.), Yanhusuo, Qingfengteng, Rehmannia glutinosa, Mulberry leaf, or a combination thereof. In one embodiment, the herbal composition the herbal composition comprises notoginseng, ginger, bornel, Ajuga nipponensis Makino, Chinese yam, or a combination thereof. In one embodiment, the herbal composition comprises honeysuckle, scutellaria baicalensis, forsythia, or a combination thereof. In one embodiment, the herbal composition comprises loptatherum gracile, mint, or a combination thereof. In one embodiment, the herbal composition comprises Honeysuckle, wild chrysanthemum, or a combination thereof. In one embodiment, the herbal composition comprises chrysanthemum, chamomile, lmperata cylindrinca, or a combination thereof.
The API may be an anti-allergy, antihistamines, antitussives, decongestants, expectorants, anti-cold/flu, analgesics, anti-inflammatories, sleep medications, anti-heartburn medications, anti-gas medications, anti-GERD medications, anti-diarrheas, laxatives, anti-smoking agent, motion sickness medications, anti-worming or anti-parasidic agent, a stimulant.

Example A PIs include, anti-allergy agents such as loratadine, diphenhydramine, cetirizine, fexofenadine, azelastine, carbinoxamine, cyproheptadine, emedastine, hydroxyzine, levocabastine, levocetirizine, chlorpheniramine, clemastine; anti-cold or flu agents such as pseudoephedrine, phenylephrine and chlorpheniramine; analgesics and anti-inflammatories such as ibuprofen, aspirin, naproxen, acetaminophen, and codeine; cough or cold medications such as guaifenesin, dextromethorphan, aminophylline, antimony potassium tartrate, atropa belladonna, belladonna alkaloids, benzonatate, camphor, caramiphen edisylate, carbetapentane citrate, hydrochloride, chlorocyclizine hydrochloride, chloropropheniramine maleate, ephedrine, its chloride, hydrochloride, sulfate; epinephrine or its bitartrate, hydrochloride, or undecylenate; eucalyptol, or euphorbia pilulifera; sleep medications such as diphenhydramine and doxylamine; heartburn medications such as ranitidine, cimetidine, famotidine, omeprazole, esomeprazole, lansoprazole, calcium carbonate and bismuth subsalicylate; anti-diarrheas such as loperamide; anti-gas agents such as simethicone; laxatives such as bisacodyl; smoking cessation agents such as nicotine; motion sickness medications such as dimenhydrinate meclizine; stimulant such as caffeine, an anti-worming/parasitic agent such as pyrantel pamoate, an anti-anxiety agent such as hydroxyzine.

Combinations of APIs may be used to treat or prevent one or more symptoms. Suitable combinations include for example: anti-allergy combinations such as loratadine/pseudoephedrine, diphenhydramine/phenylephrine, diphenhydramine/pseudoephedrine, cetirizine/pseudoephedrine, and fexofenadine/pseudoephedrine; anti-cold and flu combinations such as chlorpheniramine/phenylephrine, ibuprofen/phenylephrine, dextromethorphan/ibuprofen/phenylephrine, dextromethorphan/acetaminophen/phenylephrine, acetaminophen/phenylephrine, acetaminophen/pseudoephedrine, doxylamine/dextromethorphan/acetaminophen, acetaminophen/dextromethorphan/guaifenesin/phenylephrine; analgesics combinations such as ibuprofen/caffeine, aspirin/caffeine, naproxen/caffeine, acetaminophen/caffeine, diphenhydramine/ibuprofen, diphenhydramine/acetaminophen, diphenhydramine/naproxen, acetaminophen/caffeine/aspirin; and antitussive combinations such as guaifenesin/dextromethorphan; sleep medication combinations such as diphenhydramine/ibuprofen, diphenhydramine/aspirin, diphenhydramine/naproxen, diphenhydramine/acetaminophen; anti-gas combination such as simethicone/calcium carbonate.

In one embodiment, the gummy composition may further comprise polymers (natural, synthetic, or a combination thereof), waxes (natural, synthetic, or a combination thereof), a lipophilic composition comprising fats, fatty acids, oils, triglycerides, or waxes, elastomers, humectants, plasticizers, surfactants, emulsifiers, food acids, sweeteners, flavors, fillers or bulking agents, thickening agents, colorants, preservatives, anti-foaming agents, flavoring agents, coloring agents, pH adjusting agents, a coating, or a combination thereof.

In another aspect, the application provides methods of making the disclosed compositions.

In a third aspect, the application provides methods of using the disclosed compositions to promote health and support general wellbeing. In one embodiment, the application provides compositions for prevention and/or treatment a disorder caused by oral pathogenesis in an oral cavity. The disorder of the oral cavity is a dental plaque-related disorder. In one embodiment, the dental plaque-related disorder is gingivitis, periodontitis, caries, or sensitive teeth. In one embodiment, the disorder of the oral cavity is halitosis. In one embodiment, the disorder of the oral cavity is candidiasis.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the oral residence time comparison between a representative gummy composition as disclosed herein and a branded commercial gummy sample.

### DETAILED DESCRIPTION

The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, can be arranged, substituted, combined, separated, and designed in a wide variety of different configurations, all of which are explicitly contemplated herein. Each reference is incorporated herein by reference in its entirety.

This disclosure is generally drawn, inter alia, to compositions, methods, and processes related to gummy (i.e., gummy) composition. The composition may provide the benefit of supporting general health or treating/preventing diseases.

In one embodiment, the gummy composition comprises a gummy base and a bioactive composition. In one embodiment, the composition may further include a thickening agent, a humectant, a plasticizer, a surfactant, a defoaming or anti-foaming agent, a binder, a solvent, a sweetener, a flavoring agent, a lipophilic composition, a coloring agent, a preservative, a pH adjusting agent, or a combination thereof.

In one embodiment, the application provides the gummy composition for oral health. In one embodiment, the composition may further include a tooth hardening agent, a polishing agent, a bleaching or oxidizing agent, a peroxide stabilizer, an agent to diminish tooth sensitivity, a chelating agent, a re-mineralizing agent, a desensitizing agent, a breath freshen agent, an occlusive agent an anti-caries agent, an anti-tartar agent, anti-calculus agents, anti-bacteria agents, or a combination thereof.

### Gummy Base

The application provides a long lasting, slowly dissolving gummy composition with extended oral residence time of not less than 40 seconds. The gummy base and composition with extended oral residence time as disclosed herein has the advantage over traditional gummies of allowing for greater and prolonger contact of the bioactive composition within the oral cavity and with the oral mucosal membrane. Additionally, the gummy base and composition provide advantage over the traditional chewing gum in that chewing gum consists of polymeric ingredients that are not water soluble, whereas the gummy base and composition as provided herein may have a consistency similar to chewing gum but slowly dissolves and disappears as it is chewed eliminating the need of spitting out the chewing gum residue.

An example of components that can be used to yield slowly dissolving, long-lasting chewable gel compositions are gums. In one embodiment, the gum composition may include vegetable gums such as guar gum, xanthan gum, locust bean gum, gum Arabic (gum sudani, acacia gum, Arabic gum, gum acacia, acacia, Senegal gum, Indian gum), gum karaya, or tara gum. In one embodiment, the gum composition may include a glycoprotein.

The gelling agents are used to modulate the texture and hold the shape of the gummy composition. The gelling composition may include high molecular weight polymers capable of forming gel. Example gelling agents include starch, pectin, agar, carrageenan, alginate, gelatin, gellan, konjac, a cellulose derivative, or a combination thereof. In one embodiment, the starch comprises amylose starch or modified starch. The gelling composition may include one or more gelling agents. In one embodiment, Konjac mannan interacts synergistically with other polysaccharides gelling agent and forms thermo-reversible gels.

The gummy base may include mixtures of starch and one or more gums such as gum Arabic, locust bean gum, xanthan gum, guar gum, which emulsifies and binds them in a hydrocolloidal matrix. The starch and gum hydrocolloidal matrix reduce the rate in which the gummy dissolves allowing the extended oral resident time and therefore prolonged contact of the bioactive composition with the oral cavity and oral mucosal membrane.

Gelatin may be combined with hydrocolloids such as pectin, agar, starch, or gum Arabic to create desired textures. In one embodiment, gelatin may be combined with gum Arabic to provide chewable base.

Agar may be combined with locust bean gum to provide chewable base in which polysaccharides from agar and locust bean gum synergistically interact with each other to form a strong gel that prevent weeping of agar gels. In one embodiment, locust bean gum is combined with kappa-carrageenan to prevent water from chewable base. In one embodiment, Tara gum may be used instead of locust bean gum.

In one embodiment, the gummy composition may include one or more gums. Some gums may require a mixture of two or more components to synergically interact to yield a gel. Xanthan gum and locust bean gum is an example of two gums that yield vicious solutions independently yet synergistically yield gels when mixed. Mixtures of xanthan gum and locust bean gum reach maximum gel strength at a ratio 1:1 whereas a ratio of 1 part xanthan gum to three parts locust bean gum exhibit greater elasticity. In one embodiment, the gum composition comprises xanthan gum and locust bean gum.

In some embodiments, xanthan gum and Tara gum synergically yield gels as well as guar gum and xanthan gum. In one embodiment, xanthan gum and gellan mixtures synergistically yield gels with different properties than the gels that gellan forms on its own. The polysaccharide chains of the vegetable gums can form double helices in aqueous solution after cooling leading to the formation of gel structures due to the formation of helical structures. The helical structures cause the gum to aggregate in cool water leading to the hydrocolloid. In one embodiment, in the presence of appropriate cations, the double helices may form cation-mediated aggregates, which can lead to stronger gel networks.

TABLE 1 below lists vegetable gums useful for the present application, their usage ratio, the pH range that the gum can be used at and whether the gum has synergy with other gums.

TABLE 1
Vegetable gums and their processing conditions and synergy.

| gum | Usage Ratio | Solubility | pH | Synergy |
|---|---|---|---|---|
| Arabic/Acacia gum | 5-30% | Cold | 3-9 | No |
| Guar gum | 0.1-1.5% | Cold | 4-10 | Yes, locust bean gum, xanthan gum |
| Perfected guar gum | 0.1-1.5% | Cold, more rapidly | 4-10 | Yes, locust bean gum, xanthan gum |
| | | than traditional | | |
| Locust bean gum | 0.15-0.75% | 165 F. | 4-10 | Yes, elastic with xanthan gum |
| Konjac gum | 0.5-0.7% | cold | 3-10 | Yes, elastic with xanthan gum |
| Tara gum | 0.1-1% | 180 F. | 4-10 | No |
| Xanthan gum | 0.05-0.35% | cold | 1-13 | Yes, elastic with konjac, guar, and |
| | | | | locust bean gum |
| Perfect xanthan | 0.05-0.35% | Cold, more rapidly | 1-13 | Yes, elastic with konjac, guar, and |
| gum | | than traditional | | locust bean gum |

Mon-, di-, and tri-saccharides

The bonding composition helps to bind the gummy composition together through interaction with the gelling composition, the gum composition, or both. The bonding composition keeps the texture of the gummy composition semi-solid and chewable by acting as a humectant through hydrogen bonding or cross-linking with water molecules. By hydrogen bonding with water, the bonding composition prevents the gummy from crystalizing, from drying out, and giving the gummy its chewy texture.

The bonding composition may include sucrose, fructose, glucose, low glycemic index (GI) sugar such as trehalose (also known as mycose or tremalose), palatinose (also known as isomaltulose). Palatinose has a low insulin index of 30 and may help to stabilize the insulin level. Palatinose also has lower instances of tooth decay.

In one embodiment, the bonding composition comprises a low GI sugar having a glycemic index of not more than 35, 30, 20, 15 or 10. In one embodiment, the bonding composition is substantially free of sugar having a glycemic index of more than 35 or 20. In one embodiment, the low GI sugar comprises psicose, sorbose, tagatose, trehalose, palatinose, raffinose or a combination thereof. In one embodiment, the bonding composition consists essentially of psicose, palatinose, and a third low GI sugar selected from a group consisting of trehalose, sorbose, tagatose, or a combination thereof. In one embodiment, the bonding composition further comprises N-acetyl glucosamine.

In one embodiment, the bonding composition comprises psicose, trehalose and palatinose. In one embodiment, the bonding composition comprising psicose, trehalose and palatinose, wherein palatinose may be more than 50% of the bonding composition. In one embodiment, the bonding composition has a glycemic index of not more than 8, 10, 15 or 20.

In one embodiment, the bonding composition is substantially free of sucrose, fructose and glucose. In one embodiment, the bonding composition is substantially free of sugar alcohol.

### Sugar Alcohols

Sugar alcohols include sorbitol, mannitol, erythritol, xylitol, isomalt, maltitol, lactitol, and hydrogenated starch hydrolysates. Sugar alcohols provides the benefit of being non-cariogenic and non-caloric. Sugar alcohols may mask other flavors. For example, mannitol may mask bitterness by a mechanism that involves the endothermic nature of mannitol dissolving into water.

### Thickening Agents

A thickener may include organic and inorganic thickeners or viscosity modifiers. Organic thickeners such as natural and synthetic gums and colloids may be incorporated in the gummy composition. Examples of such organic thickeners include carrageenan (Irish moss), xanthan gum and sodium carboxymethyl cellulose, starch, polyvinylpyrrolidone, hydroxyethylpropyl cellulose, hydroxybutyl methyl cellulose, hydroxypropylmethyl cellulose, and hydroxyethyl cellulose. Inorganic thickeners include Laponite D, amorphous silica compounds, which function as thickening agents including, colloidal silica compounds available under tradenames such as Cab-o-sil fumed silica manufactured by Cabot Corporation and distributed by Lenape Chemical, Bound Brook, N.J., Zeodent 165 from J. M. Huber Chemicals Division, Havre de Grace, Md. 21078 and Sylox 15 from Grace Davison, Baltimore, Md. 21203.

In one embodiment, the thickener is selected from one or more of a natural or synthetic gum, carrageenan, hydroxymethyl cellulose, a siliceous thickener or fumed silica.

In one embodiment, the gummy composition may include crosslinked acrylic acid polymers. In one embodiment, the gummy composition may include the “carbomer” family of polymers, such as carboxypolyalkylenes, available under the Carbopol® trademark. In one embodiment, the gummy composition may include hydrophilic polymers such as polyethylene oxides, polyoxyethylene-polyoxypropylene copolymers and polyvinylalcohol; or cellulosic polymers such as hydroxypropyl cellulose, hydroxyethyl cellulose, hydroxypropyl methylcellulose, hydroxypropyl methylcellulose phthalate, and methyl cellulose.

To prepare a uniform gel, dispersing agents such as alcohol or glycerin may be added, or the polymer can be dispersed by trituration, mechanical mixing or stirring, or combinations thereof. These polymers can also act as emulsion stabilizers such as carbomers.

In one embodiment, the gummy composition may include the thickening agent in the range of from 1 to 15 w % or 2 to 4 w %.

### Humectants

Humectants are low molecular weight species that give the sensation of moisture in the gummy composition. The humectant prevents the composition from drying out and helps to maintain softness and shelf life. The humectants mimic water in the composition, which allows for low water content in the composition. In one embodiment, the water content is from 2 to 6%.

Example humectants include glycerol (glycerin, 1,2,3-propantriol), sorbitol, propylene glycol (1,2-propandiol), aloe vera gel, glyceryl triacetate, and a-hydroxyacids (lactic acid). The gummy composition may include one or a mixture of humectants. In one embodiment, the humectant comprises a mixture of glycerin and sorbitol. In one embodiment, the gummy composition may include propylene glycol and glycerin as humectants.

The humectant content may be in the range of from about of 1 to about 30 w % or about 2 to about 25 w %.

### Sweeteners

Sweetener may be non-cariogenic therefore the gummy composition is substantially devoid of cariogenic sugars such as sucrose, fructose, and glucose.

Non-cariogenic sweeteners are suitable for the oral health gummy. Example non-cariogenic sweeteners suitable includes rare sugars, sugar alcohols, and sugar substitute. Example rare sugars include psicose, sorbose, tagatose, trehalose, palatinose, or a combination thereof. Example sugar alcohol include erythritol, sorbitol, mannitol, maltitol, isomalt, and xylitol. Example sugar substitutes include saccharin, aspartame, sucralose and acesulfame K. In some embodiments, the sweetener used in the composition not only provides sweetness but also decreases the populations of bacteria in the mouth that lead to oral health problems.

In one embodiment, the sweetener is selected from saccharin, aspartame, cyclamate, sucralose, Stevia, mannitol, sorbitol, xylitol and similar glycols.

### Plasticizers

Plasticizers impart flexibility by lowering the glass transition temperature of the polymers. They act to decrease brittleness and increase chewiness of the gummy composition. Example plasticizers may include lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium and potassium stearates.

### Flavoring Agents

The gummy composition may include one or a mixture of flavoring agents. Example flavoring agents include bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, such as spearmint essential oil. Some flavoring agents can also act as sweeteners and can be use as such and include, for example, neohespiridin dehydrochalcone, xylitol, Sucralose, and mixtures thereof, such as xylitol.

Flavors and colors are for sensory appeal. Flavor components in gum exist in liquid, powder or micro-encapsulated forms. Liquid flavor agents may be water-soluble, oil-soluble, or water-dispersible emulsions.

In one embodiment, the flavoring agent may be a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

The flavoring agent content may be in the range of from 0.05 to 3 w % or 0.5 to 1 w %.

### Surfactants

The gummy composition may include one or a mixture of surfactants. The surfactant may be nonionic, anionic and amphoteric. Example surfactant include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof including, for example, sodium lauryl sulfate (SLS) or sodium lauryl ether sulfate (SLES) as well as anionic taurate surfactants including, for example, sodium methyl cocoyl taurate and sodium methyl oleoyl taurate, PEG caster oils, and PEG hydrogenated caster oils, including, for example, PEG-60 Hydrogenated Castor Oil Such as, the oral care composition embodiments include sodium lauryl sulfate (SLS) and sodium methyl cocoyl taurate, more such as, a mixture of sodium lauryl sulfate (SLS) and sodium methyl cocoyl taurate. Oil Such as, the oral care composition includes PEG-60 Hydrogenated Castor Oil.

In one embodiment, the surfactant comprises sodium lauryl sulfate, sodium N-coco, N-methyl taurate, sodium N-lauroyl sarcosine, or a compatible dental detergent. In one embodiment, the surfactant comprises lecithin, cholesterol, lanolin, tea saponin, protein, saponins, sugars, alkyl polyglycosides, or a combination thereof.

The surfactant content may be from 0.1 to 10 w % or 2 to 5 w %.

### Defoaming and Anti-Foaming Agents

A defoamer or an anti-foaming agent is a chemical additive that reduces and hinders the formation of foam. They are used to prevent formation of foam or is added to break a foam already formed. Defoamers eliminate existing foam and anti-foamers prevent the formation of further foam.

Oil based defoamers have an oil carrier. The oil might be mineral oil, vegetable oil, white oil or any other oil that is insoluble in the foaming medium, except silicone oil. An oil based defoamer may contains a wax and/or hydrophobic silica to boost the performance. Typical waxes are ethylene bis stearamide (EBS), paraffin waxes, ester waxes and fatty alcohol waxes. These products might also have surfactants to improve emulsification and spreading in the foaming medium. Powder defoamers are in principle oil based defoamers on a particulate carrier like silica. These are added to powdered products like cement, plaster and detergents. Water based defoamers are different types of oils and waxes dispersed in a water base. The oils are often mineral oil or vegetable oils and the waxes are long chain fatty alcohol, fatty acid soaps or esters. These may be used as deaerators, which means they can facilitate releasing entrained air. Silicone-based defoamers are polymers with silicon backbones. These might be delivered as an oil or a water-based emulsion. The silicone compound consists of a hydrophobic silica dispersed in a silicone oil. Emulsifiers are added to ensure that the silicone spreads fast and well in the foaming medium. The silicone compound might also contain silicone glycols and other modified silicone fluids. Polydimethylsiloxane is a widely used antifoaming agent. Silicone based defoamers may be suitable in non-aqueous foaming systems like crude oil and oil refining. For very demanding applications fluorosilicones may be suitable. EO/PO based defoamers contain polyethylene glycol and polypropylene glycol copolymers. They are delivered as oils, water solutions, or water-based emulsions. EO/PO copolymers normally have good dispersing properties and are often well suited when deposit problems are an issue. Alkyl polyacrylates are suitable for use as defoamers in non-aqueous systems where air release is more important than the breakdown of surface foam. These defoamers are often delivered in a solvent carrier like petroleum distillates.

In one embodiment, the surfactant comprises insoluble oils, polydimethylsiloxanes, silicones, alcohols, stearates, glycols, or a combination thereof.

### Binders

A binder can act a viscosity increasing agent. Example water soluble binders may include for example polyvinyl alcohols, polyvinyl pyrrolidone (PVP), polyvinyl pyrrolidone derivates and copolymers, saccharides such as D-glucose, D-fructose, sorbitol, polysaccharides such as starch, cellulose and pectin including derivatives thereof, gelatine, naturally occurring substances such as alginic acid, carrageen gum, lucost bean gum, guar gum, xanthan gum, tragacanth gum, arabic gum, karaya gum.

In one embodiment, an gummy composition may include a binder composition comprising polyvinyl pyrrolidone (PVP), cellulose gum, xanthan gum, or a combination thereof as a binder and/or thickener. The binder/thickener content may be from 0.1 to 10 w %, or 5 to 8 w %.

### Coatings

A coating can be applied to the gummy composition. In one embodiment, the coating may be non-cariogenic. In one embodiment, the coating may include non-carogenic sugars or sugar alcohols. Example non-cariogenic sugar may include psicose, sorbose, tagatose, trehalose, and palatinose. Example sugar alcohols may include erythritol, sorbitol, mannitol, maltitol, isomalt, and xylitol. Resistant starches may also be used for coating.

### Preservatives

Example preservative may include benzoic acid, sodium benzoate, methylparaben, propylparaben, sorbic acid and potassium sorbate. In one embodiment, the preservative comprises sodium benzoate.

The preservative agent may be present at levels ranging from about 0.01 to about 2 w %.

### Bioactive Composition

In one embodiment, the bioactive composition comprises a vitamin composition, a mineral composition, an amino acid composition, an antioxidant composition, an herbal composition, a probiotics composition, or a prebiotics composition.

Vitamins may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D (D2, D3, or both), vitamin E, vitamin K, K1 and K2 (i.e., MK-4, MK-7), folic acid, biotin, choline, or combinations thereof.

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, or combinations thereof.

Antioxidant may include astaxanthin, carotenoids, coenzyme Q10, flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof.

The herbal composition may include extract, powders or a combination. The extract may be water extract, ethanol extract, water/ethanol extract or other organic solvent extract. The herbal powder may be derived from various plant or animal parts including, for example, roots, leaves, stems, fruits, flowers, nuts, seeds, bulbs, skin, bone, gland, or a combination thereof.

When used in the gummy composition for oral health, the herbal extracts and powders may be absorbed deep into gum tissue by physical action of chewing and through absorption through the mucous membranes where their potent bioactivity such as antibacterial properties may provide valuable benefits in treating gum disease.

In one embodiment, the herbal composition comprises a TCM herb. The TCM herb may have anti-inflammatory, anti-microbial, anti-bacteria, anti-viral, pain relief, antiseptic, analgesic, salivary promoting, anti-ulcer activities, or a combination thereof.

Example TCM herbs include Zanthoxylum nitidum (Roxb.), Yan Husuo ((Corydalis yanhusuo W. T. Wang ex Z. Y. Su et C. Y. Wu), Qingfengteng (Sabia japonica Maxim.), Rehmannia glutinosa, Mulberry leaf, Guan Gui (cortex cinnamomic), Mentha (including for example, mint, peppermint or spearmint), Centella asiatica (L.) Urban, Pueraria lobate, Lophatherum gracile, Scutellaria baicalensis Georgi, Sarcandra glabra (Thunb.) Nakai, honeysuckle, Imperata cylindrica Beauv.var. major (Nees), C. E. Hubb., wild chrysanthemum, chamomile, licorice, ginseng, Ganoderma Lucidum Karst, Ribes nigrum L, Gingko, green tea, pearl powder, powder of mother of pearl, ginger, or a combination thereof.

For oral health application, TCM herbs may be used to alleviate oral diseases include gum diseases, reducing bleeding, treating oral cavity inflammation, reducing teeth sensitivity, alleviate pain, or promote oral health in general. Zanthoxylum nitidum (Roxb.) ( [symbol] ) may be used to relieve pain and promote blood circulation. Yan Husuo ((Corydalis yanhusuo W. T. Wang ex Z. Y. Su et C. Y. Wu, [symbol] ) may be used to promote blood circulation. Qingfengteng (Sabia japonica Maxim., [symbol] ) may be used for anti-inflammatory applications including treating rheumatism and reducing swelling. Panaxndtoginseng (Burk.) F. H. Chen ( [symbol] , [symbol] ) has the effect of removing blood stasis and hemostasis, promoting blood circulation and relieving pam. Rehmannia glutinosa ( [symbol] ) may be used to reduce inflammation, relieve pain, clearing heat and nourishing blood. Mulberry leaf ( [symbol] ) may be used to clear the inflammation. Guan Gui (cortex cinnamomi, [symbol] ) may be used to promote blood circulation, relieve pain, reduce inflammation, detoxify and reduce swelling. Mentha ( [symbol] , peppermint, spearmint) may be used to treat toothache, relieve coughing, cough, and reducing bleeding. Centella asiatica (L.) Urban ( [symbol] ) may be used to reduce inflammation, reduce swelling and detoxification. Ribes nigrum L ( [symbol] ) may be used to promote gum health and protect teeth.

Additional TCM herbs that may be incorporated into the gummy composition for general health or oral health benefits. Pueraria lobate ( [symbol] ) may be used to relieve muscles pain, reduce fever, reduce rashes, and treating diarrhea. Lophatherum gracile ( [symbol] ) is an anti-inflammatory TCM herb that may be used to treat fever, dehydration, tongue and mouth sore. It is also a diuretic. Scutellaria baicalensis Georgi ( [symbol] ) is an anti-inflammatory TCM herb that may be used to detoxify, reduce fever, reduce upper respiratory tract infection and treat cough, pneumonia, dysentery, hemoptysis, red eyes and high blood pressure. Scutellaria baicalensis has been shown to have antibacterial activities. Sarcandra glabra (Thunb.) Nakai ( [symbol] ) is an anti-inflammatory herb that may be used to reduce swelling and relieve pain. It has been shown to have antibacterial and anti-inflammatory properties. Honeysuckle may be used to clear the inflammation, detoxify, and reduce fever. The herb has anti-inflammatory and anti-viral activities. Wild chrysanthemum may be used as an antipyretic and antipyretic herb. It is used to treat cold, sore throat, eczema, and hypertension. Imperata cylindrica Beauv.var. major (Nees) C. E. Hubb. ( [symbol] ) may be used as diuretic, blood coagulant, and immune booster. In addition, it also has antibacterial effect.

In one embodiment, the herbal composition provides pain relief, teeth cleaning and gum protection property. In one embodiment, the herbal composition comprises Zanthoxylum nitidum (Roxb.), Centella asiatica, mentha ( [symbol] , [symbol] , [symbol] ), or a combination thereof. In one embodiment, the herbal composition provide pain relieve analgesic effect. In one embodiment, the herbal composition provides lastingly relieve toothache-related problems such as bleeding gums, red gums, and tooth sensitivity. In one embodiment, the herbal composition comprises Zanthoxylum nitidum (Roxb.), Yanhusuo, Guangui ( [symbol] , [symbol] ). These Chinese herbal medicines have the functions of rapid pain relief, swelling and hemostasis, and has the effects on inhibiting harmful bacteria in the oral cavity.

In one embodiment, the herbal composition provides a rapid pain relieve effect and can quickly relieve gum pain. In one embodiment, the herbal composition provides platelet aggregation and strengthen gum tissues against inflammation. In one embodiment, the herbal composition promotes microcirculation.

In one embodiment, the herbal composition provides pain relief and repairing function to the tooth, reducing swelling and hemostasis, clearing heat and regenerating, and deeply improving oral microbiota population.

In one embodiment, the herbal composition comprises Zanthoxylum nitidum (Roxb.), Yanhusuo, Qingfengteng, Rehmannia glutinosa, Mulberry leaf ( [symbol] , [symbol] , [symbol] , [symbol] , [symbol] ), or a combination thereof. In one embodiment, the herbal composition comprises notoginseng ( [symbol] ), musk ( [symbol] ) bovis calculus sativus ( [symbol] ) snack gallbladder ( [symbol] ). For oral care, the composition may be used to reduce gum problems (bleeding gums, gum pain), repairs mucosal damage, nourishes gums, and improves periodontal health.

In one embodiment, the herbal composition comprises Liangmianzheng ( [symbol] , Zanthoxylum nitidum (Roxb.) DC). The composition has the effect of anti-inflammation, detoxify, promote blood circulation, reduce swelling and relieve pain.

In one embodiment, the herbal composition can detoxify and eliminate swelling and remove harmful bacteria, inhibit bleeding, and repair wounds, relieve blood stasis and relieve pain, restore gingival teeth and solid teeth layer by layer, and balance pH value to reshape good microbiota population.

In one embodiment, the herbal composition comprises ginger. Ginger is a TCM with the functions of anti-inflammatory and fresh breath.

In one embodiment, the gummy composition is substantially void of sugar, fluorine, artificial color, fluorine, and artificial preservative. In one embodiment, the gummy composition contains active calcium and xylitol to nourishes the gums, increases calcium and strengthens teeth.

In one embodiment, the herbal composition comprises Dandelion, Isatisis (Radix Isatis), Scutellaria baicalensis, Radix Isatidis, or a combination thereof. In one embodiment, the herbal composition comprises Dandelion Root, Radix Isatis, Corydalis bungeana, Baikal Skullcap, or a combination thereof. In one embodiment, the herbal composition comprises watermelon frost, a TCM formulation made from watermelon skin and skin nitrate. In one embodiment, the herbal composition comprises honeysuckle, scutellaria baicalensis, and forsythia. It has the functions of reducing swelling, anti-inflammation and detoxifying. In one embodiment, the herbal composition comprises loptatherum gracile and mint. In one embodiment, the herbal composition comprises honeysuckle and wild chrysanthemum. In one embodiment, the herbal composition comprises while chrysanthemum, chamomile, and lmperata cylindrinca.

In one embodiment, the herbal composition comprises radix rehmanniae, fructus corni officinalis, radix dioscoreae oppositae, alismatis rhizoma, sclerotium poriae cocos and cortex moutan radicis. Tripterygium wilfordii may be used in the treatment of a wide spectrum of autoimmune and inflammatory diseases.

In one embodiment, the herbal composition comprises asparagus root, tuber ophiopogonis japonici, radix scrophulariae ningpoensis, flos Lonicerae japonicae and radix glycyrrhizae uralensis. In one embodiment, the herbal composition comprises radix ligustici chuanxiong, radix ginseng, radix paeoniae lactiflorae, semen persicae and radix et caulis jixueteng. In one embodiment, the herbal composition comprises vietnamese sophora root, bistort rhizome, north valerianaceae, cortex dictamni, Prunella vulgaris L and Dioscorea bulbifera. In one embodiment, the herbal composition can modulate immune reactions, inhibit production of inflammatory cytokines, suppress tumor cell proliferation and reduce the incidence of squamous cell carcinoma.

In one embodiment, the herbal composition comprises a TCM herb with antimicrobial activity. Example TCM herbs include Fructus armeniaca mume, Porphyromonas gingivalis, including Cortex magnoliae officinalis, Cortex phellodendri, Flos caryophylli, Flos lonicerae japonicae, Fructus armeniaca mume, Fructus forsythiae suspensae, Herba cum radice violae yedoensitis, Herba menthae haplocalycis, Pericarpium granati, Radix et rhizoma rhei, Radix gentianae, Ramulus cinnamomi cassia and Rhizoma cimicifugae. In one embodiment, the herbal composition comprises Cortex phellodendri. In one embodiment, the herbal composition comprises Radix et rhizoma rhei. In one embodiment, the herbal composition comprises Fructus armeniaca mume.

In one embodiment, the herbal composition comprises Aloe Vera. Aloe vera has a soothing and anti-inflammatory effect. While not directly addressing any particular oral problem, its anti-inflammatory effects address the swelling brought on by many oral health issues.

In one embodiment, the herbal composition comprises Calendula officinalis. Calendula officinalis has soothing, anti-inflammatory properties that help heal irritated, tender gums. It also has a mild antiseptic action. Similar to aloe vera in not directly addressing any particular oral problem, its anti-inflammatory and antiseptic effects address the swelling brought on by many oral health issues.

In one embodiment, the herbal composition comprises echinacea. Extracts of echinacea are immune stimulating, which helps combat infection; it is anti-inflammatory and antiviral.

In one embodiment, the herbal composition comprises Goldenseal. Goldenseal (Hydrastis canadensis) has anti-inflammatory, antiviral and antibiotic properties; it's good for healing and preventing gum problems.

In one embodiment, the herbal composition comprises grapefruit. Grapefruit seed extract and freeze-dried pulp powders, made from the seeds and pulp of grapefruit, has antiseptic and antibacterial properties to fight infection.

In one embodiment, the herbal composition comprises horsetail. The extracts of horsetail are high in concentration in silica, vitamin E, selenium and other minerals. When used in the oral health chewable, it can help in coagulation, decrease bleeding and heal oral infections.

In one embodiment, the herbal composition comprises myrrh. Myrrh (Commiphora molmol) has antimicrobial and astringent properties that help to tighten the gums. When used for the oral health, it aids in healing bleeding gums and mouth ulcers, and helps fight the bacteria that cause tooth decay and gum disease.

In one embodiment, the herbal composition comprises neem. Neem (Azadirachta indica) helps prevent the diseases associated with plaque buildup including gingivitis and dental caries.

In one embodiment, the herbal composition comprises Oregon grape root. Oregon grape root (Mahonia spp.) has a high concentration of antimicrobial compounds and astringent properties, which, when may be used to prevent and modulate gum problems.

In one embodiment, the herbal composition comprises sage. Sage (Salvia officinalis) has strong astringent properties, making it useful for tightening the gums and soothing a sore mouth.

In one embodiment, the herbal composition comprises white oak bark. White oak bark (Quercus alba) is a strong astringent that is helpful for healing swollen, tender and bleeding gums and mucous membranes, and has a clotting and antiseptic effect.

In one embodiment, the herbal composition comprises turmeric or curcumin. Turmeric, especially curcumin, promotes oral health problems as documented in the following: 1. Clinical efficacy of turmeric use in gingivitis: A comprehensive review, Stoyell K A, Mappus J L, Gandhi M A, Complement Ther Clip Pract. 2016 November; 25: 13-17). [0006] 2. Efficacy of curcumin in the treatment of chronic gingivitis: a pilot study. Muglikar, S., Patil, K. C., Shivswami, S., and Hegde, R., Oral Health and Preventive Dentistry. 2012; 11(1):81-86) [0007] 3. comparison of effectiveness of curcumin with triamcinolone acetonide in the gel form in treatment of minor recurrent aphthous stomatitis: A randomized clinical trial., Deshmukh R A, Bagewadi A S, Int J Pharm Investig. 2014 July; 4(3):138-41). [0008] 4. Oral care compositions, Brading et al., U.S. Pat. No. 8,916,139.

In one embodiment, the herbal composition comprises tea. Chen et al. further indicated the abilities of epigallocatechin-3-gallate, luteolin, apigenin, myricetin, quercetin, and cyanidin in inhibiting retinal pigment epithelial cells in “Effects of the vegetable polyphenols epigallocatechin-3-gallate, luteolin, apigenin, myricetin, quercetin, and cyanidin in primary cultures of human retinal pigment epithelial cells” (Molecular Vision, 2014, 20:242-258). More studies indicated the abilities of epigallocatechin-3-gallate in inhibiting the proliferation of aortic smooth cells: (Z. Shu, M. Yu, G. Zeng, X. Zhang, L. Wu, X. Tan, 2014, “Epigallocatechin-3-gallate inhibits proliferation of human aortic smooth muscle cells via up-regulating expression of mitofusin,” European Journal of Cell Biology, 93:137-144; P. L. Liu, J. T. Liu, H. F. Kuo, I.

W. Chong, and C. C. Hsieh, “Epigallocatechin Gallate Attenuates Proliferation and Oxidative Stress in Human Vascular Smooth Muscles Cells Induced by Interleukin-1.beta. via Heme Oxygenase-1,” 2014, Mediators of Inflammation, Article ID 523684.

Fibroblasts play crucial roles during wound healing. When the existence of a wound is detected, fibroblasts will be activated to become polygonal phenotype as discussed by Li Y C et al. in “Pearl extract enhances the migratory ability of fibroblasts in a wound healing model” (Pharm. Biol., 2013, 51:289-297). When the wound is healing, fibroblasts will proliferate and move to the location of the wound for repairing as discussed by Khovidhunkit, S. O. et al. in “In vitro study of the effects of plaunotol on oral cell proliferation and wound healing” (J. Asian Nat. Prod. Res., 2011, 13:149-159). The increased quantity of fibroblasts improved the condition of wound healing in experimental models as discussed by Lamme E. N. et al. in “Higher numbers of autologous fibroblasts in an artificial dermal substitute improve tissue regeneration and modulate scar tissue formation” (J. Pathol., 2000, 1900:595-603).

Herbal actives may include polyphenol, flavonoids, allied phenolic compounds, polyphenolic compounds, terpenoids, alkaloids, sulphur-containing compounds, polysaccharides, flavone, flavonoids, quinone, or combinations thereof.

In one embodiment, the herbal composition comprises plant polyphenol effective in improving the proliferation of fibroblasts. In one embodiment, the polyphenol comprises a citrus polyphenol. In one embodiment, the citrus polyphenol includes delphinidin, pelargonidin, peonidin, malvidin, cyaniding, narirutin, naringin, hesperidin, and neohesperidin.

Herbal actives may be essential oils. EOs may contain aromatic molecules that rapidly absorb through the oral mucous membranes. When used in dental care, EOs are absorbed deep into gum tissue and oral mucous membranes where their potent antibacterial properties provide valuable benefits in the treatment of gum disease. EOs have the additional function of providing flavor for the oral health chewable.

Some EOs used for the oral health chewable are the following:

Red Thyme. The oil from this herb has been used extensively in medicine as a antiseptic and disinfectant. EOs from red thyme have antibacterial, antifungal, anti-inflammatory, antimicrobial and stimulating properties that help treat oral inflammation and infection.

Cinnamon Bark. The high aldehyde content of this oil results in it being an antimicrobial and antiseptic. Cinnamon has a tannins concentration resulting in cinnamon bark being an astringent. Astringents contract, firm and strengthen oral tissue, reduce surface inflammation and irritation, and create a protective barrier from infection.

Eucalyptus. Eucalyptus has antibacterial, antifungal, antiseptic and stimulant properties, which boost circulation and speed up the healing process and combats oral infection and mouth ulcers.

Lavender. The EO from this highly aromatic flower offers antibacterial, anti-inflammatory, antimicrobial, antiseptic and stimulant properties that result in the EO being a natural dental remedy, making it a strong ally for good oral health. Lavender is effective against bad breath (halitosis), helps heal damaged tissue and provides soothing relief from mouth pain.

Peppermint. The EO from this plant provides antibacterial, anti-inflammatory, antifungal, antimicrobial, antiseptic, sedative and stimulant properties. Peppermint EO used in the oral health chewable offers protection from oral infection, pain relief and enhanced blood circulation which leads to increased healing rates.

Tea tree. Tea tree (Melaleuca alternifolia) is a potent antimicrobial EO which fights the bacteria that cause tooth decay and gum disease. It is often used in combination with other natural herbs, such as rosemary, chamomile, echinacea, aloe and fresh mint and when used in the oral health chewable imparts anti-cavity and anti-gingivitis properties to the chewable.

### Probiotics

Probiotics have many positive influences in creating better heath or oral health. Probiotics have both direct and indirect interactions. Basically, probiotics help in binding oral microorganisms to proteins & biofilm formation. They fight against plaque formation and on its complex ecosystem by compromising and intervening with bacterial attachments. Through its direct interactions, probiotics compete with oral microorganisms of substances available. This process is the involvement of metabolism of substrate.

Probiotics produce chemicals to inhibit oral harmful bacteria that damage oral hygiene. On the other hand, the indirect interactions of probiotics are effective in the process of removing harmful bacteria and stabilizing normal conditions. Probiotics modulate and systematize immune function on local community as well as non-immunologic defense mechanisms. Probiotics may regulate permeability and also to develop colonies in oral microflora with less pathogenic species. Probiotics have proved to be effective in curing diseases such as dental caries, periodontal diseases, halitosis and candidiasis.

Example probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

Example oral health probiotics strains may include S. Salivarius, L. rhamnosus GG, L. acidophilus, L. casei, L. reuteri, Bifidobacterium, DN-173010, Propionibacterium freudenreichii ssp. Shermanii JS, L. rhamosus, L. paracasei, L. johnsonii, L., W. cibaria, L. casei Shirota, Lacidophilus, L., or any combination thereof.

Lactobacillus plantarum CECT 7481 and Lactobacillus brevis CECT 7480 display a significant inhibitory activity against a broad number of pathogens of the oral cavity that are implicated in the development of oral disorders, such as gingivitis, periodontitis, caries and halitosis, while displaying minimal antagonism against common commensal strains of the human oral flora. These two strains show a lack of inhibitory activity between them, thus allowing their combined use in a single formula. Furthermore, the combination of these strains has the advantage of displaying a higher antagonistic activity against oral pathogens as compared to the activity of the individual strains used separately.

A probiotic L. reuteri ATCC 55730 strain and has demonstrated to reduce gingivitis in a clinical trial (Twetman S, et al. “Short-term effect of chewing gums containing probiotic Lactobacillus.sub.-reuteri on the levels of inflammatory mediators in gingival crevicular fluid”. Acta Odontol Scand, 2009, vol. 67, p. 19-24). This same strain, L. reuteri ATCC 55730, has been reported to exert a strong antagonistic activity against cariogenic

Streptococcus salivarius K12 is another commercial probiotic intended for use in the oral cavity. S. salivarius K12 has been shown to perform in vitro antimicrobial activity against various bacterial species incriminated in the etiology of halitosis (Burton J P, et al., “Preliminary study of the effect of probiotic Streptococcus salivarius K12 on oral malodor parameters”. J Appl Microbiol, 2006, vol. 100, p. 754-764).

By antagonising microorganisms that are implicated in pathological conditions in the oral cavity, these strains have the effect of altering the oral microbiological profile to a healthier profile, thereby benefiting oral health conditions. In one embodiment, an oral probiotic may have the ability to adhere to and colonize surfaces in the oral cavity.

The term “effective amount” as used herein is the amount of colony forming units (cfu) for each strain in the composition that is high enough to significantly modify the condition to be treated in a positive way but low enough to avoid serious side effects (at a reasonable benefit/risk ratio), within the scope of sound medical judgment. An effective amount of said probiotic microorganism will be determined by the skilled in the art and will vary with the particular goal to be achieved, the age and physical condition of the patient being treated, the severity of the underlying disorder, and the final formulation. For instance, in oral health products, the strain or strains are present in an amount from about 10.sup.5 cfu/g to about 10.sup.12 cfu/g, such as in an amount from about 10.sup.7 cfu/g to about 10.sup.11 cfu/g. The term “colony forming unit” (“cfu”) is defined as number of bacterial cells as revealed by microbiological counts on agar plates. In a particular embodiment, the composition of the application is an oral care product comprising between 10.sup.7-10.sup.10 cfu/g.

In one embodiment, the gummy composition contains probiotic strains in an amount ranging from 10.sup.5 and 10.sup.12 cfu/g. In a particular embodiment, the composition of the application is a dietary supplement comprising between 10.sup.7-10.sup.10 cfu/g.

### Prebiotics

Prebiotics may be used to complement probiotics in the treatment of oral diseases. Their function is to enhance the growth and activity of beneficial organisms and simultaneously suppress the growth and activity of potentially deleterious bacteria. In this way prebiotics modify the balance of the intestinal micro-flora.

Example prebiotics useful for the application include Lactose, Inulin, Fructo oligosacccharides, Galacto oligosaccharides and Xylo oligosaccharides.

Prebiotics when combined with probiotics have many advantages. Prebiotics may selectively stimulate the growth of probiotics, which is dose and strain dependent.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

### Polishing Agents

In one embodiment, the gummy composition includes a polishing agent for oral care. The polishing agent may include hydrated silica, calcium, bentonite, kaolin clay, pearl powder, the powder of mother of pearl, nutshell powder, salt, or a combination thereof.

The polishing agent can, for example, calcium carbonate, sodium metaphosphate, potassium metaphosphate, tricalcium phosphate, di-hydrated dicalcium phosphate, calcined alumina and siliceous materials, bentonite clay, kaolin clay or combinations thereof. Example abrasives include calcium carbonate, siliceous materials, such as silica and more such as hydrated silica, bentonite clay, kaolin clay or combinations thereof. More such as abrasives include a mixture of calcium carbonate, bentonite clay, kaolin clay and hydrated silica. In one embodiment, the polishing agent may be selected from one or more of finely divided silica, calcium carbonate, tricalcium phosphate, dicalcium phosphate and insoluble sodium metaphosphate.

The abrasive content for the oral care gummy composition may be in the range of from 1 to 80 w % or 35 to 50 w %.

### Oxidizing or Bleaching Agents

In one embodiment, the gummy composition comprises an oxidizing or bleaching agent. In one embodiment, the oxidizing agent is peroxide or its derivatives.

In one embodiment, the peroxide may include hydrogen peroxide which is present in an amount of from 0.5 to 35 w %.

Examples include hydrogen peroxide, peroxydiphosphate, urea peroxide, metal peroxides such as calcium peroxide, sodium peroxide, stronthium peroxide, magnesium peroxide, and the salts of perborate, persilicate, perphosphate and percarbonate such as sodium perborate, potassium persilicate and sodium percarbonate. The oxidizing agent content the oral care composition embodiments can be in the range of from 1 to 35 w % or 5 to 14 w %.

In one embodiment, the oral composition comprises hydrogen peroxide employed at concentrations of from 0.5 w % to 35 w %, or 5 to 14 w %.

### Peroxide Stabilizers

An oral or pharmaceutically acceptable bleaching agent or peroxide stabilizer can include one or a mixture of such agents, such as, for example, ethylenediamineteraacetic acid, disodium salt; ethylenediamineteraacetic acid, tetrasodium salt; ethylenediaminetetraacetic acid, calcium disodium salt; etidronic acid; citric acid; gluconic acid; sodium citrate; sodium gluconate; sodium phosphate; disodium phosphate; trisodium phosphate; tetrapotassium pyrophosphate; sodium tripolyphosphate as well as sodium stannate and potassium stannate (tin can be a or peroxide stabilizer). The peroxide stabilizer content the oral composition embodiments can be in the range of from about of 0.01 to 2 w % and such as 0.05 to 0.3 w %.

### Anti-Caries Agents

Embodiments of the present disclosure can also include sources of fluoride ions, or fluorine-providing compounds useful, for example, as anti-caries agents known for their ability to release fluoride ions in water. An oral or pharmaceutically acceptable fluoride source can include one or a mixture of fluoride source, such as, for example, fluoride salts, such as soluble alkali metal and alkaline earth metal salts, for example, sodium fluoride, potassium fluoride, ammonium fluoride, calcium fluoride, copper fluorides such as cuprous fluoride, zinc fluoride, barium fluoride, sodium fluorosilicate, ammonium fluorosilicate, sodium fluorozirconate, sodium monofluoro-phosphate, aluminum mono- and di-fluorophosphate, and fluorinated sodium calcium pyrophosphate. Alkali metal and tin fluorides, such as sodium and stannous fluoride, sodium monofluorophosphate, and mixtures thereof, are example. It is example to have the fluoride source in the oral care composition at concentrations ranging from 0.005 to 3 w %.

### Tooth Hardening Agents

In one embodiment, the gummy composition for oral health comprises a tooth hardening agent. In one embodiment, the tooth hardening agent is selected from soluble alkali metal fluorides, sodium fluoride, potassium fluoride; copper fluoride, tin fluorides, ammonium fluorosilicate, sodium fluorozirconate, ammonium fluorozirconate, sodium monofluorophosphate, aluminum fluorophosphates (mono-, di- and tri-), fluorinated sodium calcium pyrophosphate, sodium monofluorophosphate and mixtures thereof,

### Anti-Tartar Agent

In one embodiment, the gummy composition for oral health comprises an anti-tartar agent. Example anti-tartar agent includes polyphosphates, alkali metal tripolyphosphates, alkali metal pyrophosphates and sodium pyrophosphate.

### Anti-Calculus Agent

In one embodiment, the gummy composition for oral health comprises an anti-calculus agent. Example anti-calculus agent include an azacycloalkane diphosphonic compound, azacycloheptane diphosphonic acid and salts thereof, synthetic anionic polymeric polycarboxylates, and copolymers of maleic acid or maleic anhydride with vinyl methyl ether, and their salts.

### Re-Mineralizing Agents

Calcium glycerophosphate is a dental agent is capable of re-mineralizing enamel. Example re-mineralizing agent can include a phosphate compound, a calcium compound, a calcium phosphate compound, hydroxyapatite and a caseinate. The phosphate compound can include, for example, a monobasic phosphate compound, a dibasic phosphate compound, a tribasic phosphate compound, calcium glycerophosphate, and combinations thereof.

### Desensitizing Agents

Desensitizing agent may be used to diminish teeth sensitivity for the oral care application. Example desensitizing agents may include one or a mixture of such agents, such as, for example, strontium chloride, potassium nitrate and potassium citrate. The agent used to diminish teeth sensitivity content can be in the range of from about of 0.1 to 5 w %. A example agent in the oral care composition embodiments is potassium nitrate.

### pH Adjusting Agents

pH adjusting agent may be used to modify the pH of the gummy composition. Example pH adjusting agents include sodium bicarbonate, sodium hydroxide, ammonium hydroxide, calcium glycerophosphate, triethanolamine (an organic compound composed of a tri-alcohol & an amine) or a combination thereof. In one embodiment, sodium bicarbonate can be employed as a solution at concentrations of from 5 to 99.5 w %, from 10 to 80 w % or as a powder. In one embodiment, a mixture solution of sodium bicarbonate and sodium hydroxide may be used, wherein sodium bicarbonate the concentrations may be from 15 to 79 w % and sodium hydroxide may be at concentrations from 1 to 5 w %.

### Breath-Freshen Agents

An anti-bad-breath agent may be used for treating bad breath (halitosis). Examples anti-bad-breath agents may include cetylpyridinium chloride (CPC), zinc compounds (such as zinc chloride, zinc citrate and Zinc gluconate), and chlorhexidine (such as Zinc Chloride). The anti-bad-breath agents content can be from 0.05 to 5 w % and such as 0.075 to 2 w %.

### Chelating Agents

In one embodiment, the chelating agent is selected from phosphates, pyrophosphates, tripolyphosphates, and hexametaphosphates. Example chelating agent useful for the application include sodium tripolyphosphate, ethylenediamine tetracetic acid (“EDTA”) and its salts (e.g., tetrasodium EDTA and calcium EDTA), or a combination thereof. In one embodiment, the chelating agent may be at concentrations of from about 0.3 to about 30 w %.

In one embodiment, the chelating agent may be ethylenediamine tetraacetic acid (EDTA), ethylene glycol tetraacetic acid (EGTA), cyclohexanediamine tetraacetic acid (CDTA), hydroxyethylethylene diamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ArPA), citric acid, acetic acid and acceptable salts thereof, and any combinations thereof.

In one embodiment, the EDTA salt is selected from diammonium EDTA, disodium EDTA, dipotassium EDTA, triammonium EDTA, trisodium EDTA, tripotassium EDTA, tetrasodium EDTA, tetrapotassium EDTA, calcium disodium EDTA, and combinations thereof.

In one embodiment, the chelating agent is a polyamine selected from cyclam (1,4,7,11-tetraazacyclotetradecane), N—(C.sub.1-C.sub.30 alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4-[(5-aminopentyl)(hydroxy)am-ino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbam-oyl) propanoylamino]pentyl]-N-hydroxy-butane diamide), desferrioxamine B, desferoxamine B, DFO-B, DFOA, DFB, desferal, deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

In one embodiment, the chelating agent is a EDTA-4-aminoquinoline conjugate selected from ([2-(Bis-ethoxycarbonylmethyl-amino)-ethyl]-{[2-(7-chloro-quinolin-4-ylam-ino)-ethylcarbamoyl]-methyl}-amino)-acetic acid ethyl ester, ([2-(Bis-ethoxycarbonylmethyl-amino)-propyl]-{[2-(7-chloro-quinolin-4-yla-mino)-ethylcarbamoyl]-methyl}-amino)-acetic acid ethyl ester, ([3-(Bis-ethoxycarbonylmethyl-amino)-propyl]-{[2-(7-chloro-quinolin-4-yla-mino)-ethylcarbamoyl]-methyl}-amino)-acetic acid ethyl ester, ([4-(Bis-ethoxycarbonylmethyl-amino)-butyl]-{[2-(7-chloro-quinolin-4-ylam-ino)-ethylcarbamoyl]-methyl}-amino)-acetic acid ethyl ester, ([2-(Bis-ethoxymethyl-amino)-ethyl]-{[2-(7-chloro-quinolin-4-ylamino)-eth-ylcarbamoyl]-methyl}-amino)-acetic acid ethyl ester, ([2-(Bis-ethoxymethyl-amino)-propyl]-{[2-(7-chloro-quinolin-4-ylamino)-et-hylcarbamoyl]-methyl}-amino)-acetic acid ethyl ester, ([3-(Bis-ethoxymethyl-amino)-propyl]-{[2-(7-chloro-quinolin-4-ylamino)-et-hylcarbamoyl]-methyl}-amino)-acetic acid ethyl ester, ([4-(Bis-ethoxymethyl-amino)-butyl]-{[2-(7-chloro-quinolin-4-ylamino)-eth-ylcarbamoyl]-methyl}-amino)-acetic acid ethyl ester.

In one embodiment, the chelating agent is a tetrasodium salt of iminodisuccinic acid. In one embodiment, the chelating agent is a salt of poly-asparatic acid. In one embodiment, the chelating agent is a tetra sodium salt of L-glutamic acid N,N-diacetic acid. In one embodiment, the chelating agent is a natural chelator selected from citric acid, phytic acid, lactic acid, acetic acid and their salts and curcumin.

### Occlusive Agents

An occlusive agent may include petrolatum, mineral oil, beeswax, silicone oil, lanolin and oil-soluble lanolin derivatives, saturated and unsaturated fatty alcohols such as behenyl alcohol, hydrocarbons such as squalane, and various animal fats and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

The occlusive agent content the oral care composition may be from 1 to 35 w %, 1 to 10 w %. In one embodiment, the occlusive agent comprises coconut oil is for the oral care application.

The disclosed gummy composition may be a gummy piece in any desirable shape. Example shapes include a bar, a cylinder, a sphere, a cube, a star, a prism, a disc, gum drop, an animal shape, a flower shape, a cartoon shape, or combinations thereof.

The gummy composition may contain any pharmaceutically acceptable excipients. Selection of the excipients and the most appropriate methods for formulation in view of the particular purpose of the composition is within the scope of the person skilled in the art of pharmaceutical technology.

### EXAMPLES

### Example 1. Methods and Materials: Texture Profile Analysis (TPA)

The mechanical properties of selected functional gummies are examined with Texture Profile Analysis (TPA). TPA replicates the actual chewing experience. A TPA was performed on sample gummy with a texturometer device (TA-XT2i Plus Texture Analyser, Stable Micro Systems, U.K.) with a flat 50 mm diameter probe. The samples were compressed twice at a rate of 10 mm s-1 with a 20 mm return distance to allow the sample to deform without being penetrated. The testing parameters were: 24±2° C.; two consecutive cycles of 50% compression; crosshead at a constant speed of 30 mm min-1 and a contact force of 10.0 g. The recorded textural parameters are (1) hardness (N): Force required to compress the material by a given amount; (2) springiness (mm): elastic recovery that occurs when the compressive force is removed; (3) gumminess (N); the energy required to break down a semi-solid food before it can be swallowed; (4) chewiness (N mm); the energy required to chew a gummy into a state for swallowing; and (5) Cohesiveness; strength of the internal bonds in the sample.

### Example 2. Methods and Materials: Determination of Dissolving Time

The tablets were placed in each of the six tubes of the basket of the disintegration apparatus using water as the immersion fluid. The test was carried out for 120 minutes. The dissolving time was noted when no residue of the sample gummy, remained on the screen of the apparatus.

### Example 3. Gummy Base with Extended Residence Time

The bioactive agents are combined and added to warm water at 90° C.

Gelling or thickening agent is mixed with non-cariogenic sweetener and added to the water solution. Soak with low heat and stirring until the gelling agent dissolves. Strain through a fine sieve to remove foreign material.

A syrup mixture is created by mixing non-cariogenic sweetener, humectant, and water and heated to 124° C. The syrup mixture is poured into the gummy solution and mixed gently.

Essential oils, antigingivitis agents, waxes, surfactants and vitamins are added to the gummy base with gentle stirring.

The gummy base is added to molds and allowed to dry at 49° C. until a water content of 4-5% is reached.

For oral health formulations, non-cariogenic sweetener and teeth whitener are blended together and are applied to the surface of the molded oral health gummy.

### Example 4. Gummy Composition with Extended Oral Resident Time

Gelling agents, gum or both are added to a heated kettle equipped with stirring mechanism and abrasive (optional), defoamer (optional) and color. These components are thoroughly mixed. Water is added until 50% solids. The solution is heated to 100 C and reduced until 68% solids.

The mono and di saccharides are measured into a kettle with zinc citrate. Water is added until 65% solids. The solution is heated until 93% solids.

The two kettles are combined by the addition of the saccahride solution to the gelling solution. The system is heated to 105 C until the system is 84% solids as measured by refractometry. Flavor is added.

The long lasting chewable is then poured into molds that are either silicone, starch, or sugar alcohol or other non-cariogenic media and allow to cool. The long lasting chewable were then allowed to dry.

Formulation A: water 17.0%; gum Arabic 21.3%; glycerol 2.9%; xylitol 29.1%; maltitol 29.1%; Arginine 0.63%; Peppermint flavor 0106%; blue coloring 0.001%

Formulation B: water 17.0%; gum Arabic 29.7%; glycerol 2.9%; xylitol 24.9%; maltitol 24.8%; zinc citrate 0.63%; peppermint flavor 0.065%; spirulina 0.001%.

Formulation C: water 17.0%; gum Arabic 34.5%; glycerol 0.64%; xylitol 23.6%; maltitol 23.5%; cetirizine 0.62%; mint oil 0.065%; spirulina 0.001%.

Formulation D: water 17.0%; gum Arabic 47.4%; glycerol 0.64%; xylitol 17.8%; maltitol 16.5%; caffeine 0.303%; vitamin B complex 0.142%; peppermint flavoring 0.065%; blue coloring 0.001%.

Formulation E: water 17.0%; gum Arabic 60.4%; glycerol 0.65%; xylitol 11.3%; maltitol 10.1%; encapsulated caffeine 0.445%; pineapple extract 0.065%; beta-carotene 0.001%.

### Example 4. Gummy Composition with Extended Oral Resident Time

Gelling agent is added to a heated kettle equipped with stirring mechanism and abrasive (optional), defoamer (optional) and color. These components are thoroughly mixed. Water is added until 50% solids. The solution is heated to 100 C and reduced until 68% solids.

The mono and di carbohydrates are measured and gelling agent, actives (optional) and zinc citrate are added. These are mixed thoroughly. Water is added to a heated kettle and brought to the boiling point.

The mixture of gelling agents and carbohydrates and other actives is then added to the boiling water and mixed until uniform. The solution is heated until 78% solids as measured by refractometry.

The two kettles are combined by the addition of the carbohydrate-gelling agent solution to the gelling agent solution. The system is heated to 105 C until the system is 84% solids as measured by refractometry. Flavor is added.

The long lasting chewable is then poured into molds that are either silicone, starch, or sugar alcohol or other non-cariogenic media and allow to cool. The long lasting chewable were then allowed to dry

Formulation F: water 17.0%; gum Arabic 34.5%; carrageenan 1.9%; glycerol 0.64%; xylitol 22.6%; maltitol 22.6%; zinc citrate 0.626%; peppermint flavor 0.065%; FD&C blue 0.001%.

Formulation G: water 17.0%; gum Arabic 38.9%; gelatin 3.22%; glycerol 0.64%; xylitol 20.5%; maltitol 19.1%; arginine 0.303%; vitamin D 0.142%; peppermint flavor 0.065%; spirulina 0.001%.

Formulation H: Water 17.000; Gum Arabic 38.927%; Gelatin 3.228%; Glycerol 0.646%; Xylitol 20.516%; Maltitol 19.173%; Loratadine 0.303%; Vitamin D 0.142%; Orange Flavor 0.065%; Orange coloring 0.001%.

Formulation I: Water 17.0%; Gum Arabic 38.92%; Gelatin 6.45%; Glycerol 0.64%; Xylitol 18.90%; Maltitol 17.55%; Zinc Citrate 0.30%; Vitamin D 0.14%; Peppermint Flavor 0.064%; Beta-carotene 0.001%.

Formulation J: Water 17.00%; Gum Arabic 38.92%; Gelatin 6.45%; Glycerol 0.64%; Xylitol 18.90%; Maltitol 17.55%; Honeysuckle extract 0.30%; Chrysanthemum extract 0.14%; Citrus Flavor 0.06%; Orange coloring 0.001%.

Formulation K: Water 17.00%; Gum Arabic 27.63%; Gelatin 9.68%; Glycerol 0.64%; Xylitol 22.98%; Maltitol 21.62%; Zinc Citrate 0.30%; Vitamin D 0.14%; Peppermint Flavor 0.06%; Glyceryl mono- and di-oleates 0.03%; Coloring 0.001%.

Formulation L: Water 17.00%; Gum Arabic 38.92%; Gelatin 6.45%; Glycerol 0.64%; Xylitol 18.90%; Maltitol 17.55%; notoginseng 0.30%; ginger 0.14%; Peppermint Flavor 0.06%; Glyceryl mono- and di-oleates 0.03%; FD&C Blue 0.001%.

Formulation M: Water 17.00%; Gum Arabic 34.53%; Sodium Alginate 1.29%; Glycerol 0.64%; Xylitol 23.24%; Maltitol 22.59%; diphenhydramine 0.62%; Peppermint Flavor 0.06%; Spirulina 0.001%.

### Example 5. Comparison of Oral Residence Time Trial

Ten (10) health volunteers including an 8 female and 12 males between the age of 18 to 50 were recruited to test oral residence time of the gummies. Each volunteer was provided 3 Test gummies of the presently disclosed composition and 3 Branded commercial gummies. The branded commercial gummy has the same weight per piece as the sample testing gummies.

Each volunteer was asked to consume the gummies and track the chewing time with a digital timer. Each volunteer was asked to consume the Test gummy and the Branded gummy in alternative sequence. The timer was started at the starting of the chewing action and stopped when the gummy was completely dissolved with no discernable chunks left in the oral cavity. Each volunteer was asked to thoroughly rinse the oral cavity and rest for 15 mins between chewing the next gummy. Each volunteer recorded a total of 6 oral residence time: 3 for Test gummy, and 3 for the Branded gummy. The 3 oral residence time was averaged.

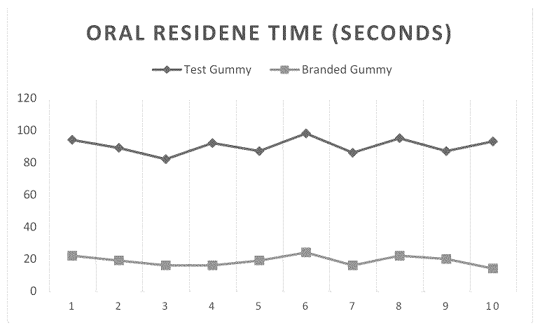

For the branded commercial gummies, the average chewing time is 18.8 seconds; in comparison, the Test gummies provided an average chewing time of 90.3 seconds, 4.7 longer than the branded commercial gummies. FIG. 1 showed the oral residence time comparison between Test gummies and Branded commercial gummies.

The use of the term “include” are to be constructed to mean “include without limitation”.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as” or “for example”) provided herein, is intended merely to better illuminate the embodiments and does not pose a limitation on the scope of the claims unless otherwise stated. No language in the specification should be construed as indicating any non-claimed element as essential.

The term “pharmaceutically acceptable” as used herein refers to compounds, materials, compositions, and/or dosage forms which are, within the scope of sound medical judgement, suitable for use in contact with the tissues of a subject (either a human or non-human animal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, excipients, etc. can be found in standard pharmaceutical texts.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present application. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

1. A gummy composition, comprising a semi-solid chewable base comprising a gum composition, a gelling composition, and a bonding composition, wherein the gelling composition comprises a protein-based gelling agent, a carbohydrate-based gelling agent, or a combination thereof, wherein the gum composition and the gelling composition have a ratio of not more than 4:1 w/w, and wherein the bonding composition comprises a mono-, di- tri-, or oligosaccharide, a sugar alcohol, or a combination thereof.

2. The gummy composition of claim 1, wherein the gum composition comprises a plant gum, a glycoprotein, or a combination thereof.

3. The gummy composition of claim 2, wherein the plant gum comprises xanthan, guar gum, locust bean gum, gum Karaya, gum Tragacanth, Tara gum, gum Arabic, or a combination thereof.

4. The gummy composition of claim 1, wherein the gum composition comprises at least two plant gums.

5. The gummy composition of claim 1, wherein the gum composition comprises gum Arabic.

6. The gummy composition of claim 1, wherein the protein-based gelling agent comprises gelatin, egg white protein, albumin, or a combination thereof.

7. The gummy composition of claim 1, wherein the carbohydrate-based gelling agent comprises starch, pectin, carrageenan, alginate, agar, gellan, konjac, a cellulose derivative, or a combination thereof.

8. The gummy composition of claim 7, wherein the starch comprises corn starch, tapioca starch, arrowroot starch, potato starch, wheat starch, yam starch, mung bean starch, rice starch, bracken starch, sago, modified starch, pre-gelatinized starch, or a combination thereof.

9. The gummy composition of claim 7, wherein the cellulose derivative comprises methylcellulose (MC), hydroxypropyhnethyl cellulose (HPMC), hydroxyethylmethyl cellulose (HPMC), hydroxyethyl cellulose (HEC), carboxynethyleellulose (CMC), or a combination thereof.

10. The gummy composition of claim 1, wherein the gelling composition comprises a protein-based gelling agent.

11. The gummy composition of claim 1, wherein the gum composition comprises gum Arabic and the gelling composition comprises gelatin.

12. The gummy composition of claim 1, wherein the gum composition and the gelling composition have a ratio from about 4:1 w/w to about 1:2 w/w.

13. The gummy composition of claim 1, wherein the bonding composition comprises sucrose, glucose, fructose, L-fructose, L-glucose, L-galactose, psicose, palatinose, trehalose, tagatose, sorbose, D-maltose (1,4-diglucose), raffinose, or a combination thereof.

14. The gummy composition of claim 1, wherein the bonding composition consists essentially of psicose, palatinose, and a third sugar selected from a group consisting of trehalose, sorbose, tagatose, or a combination thereof.

15. The gummy composition of claim 1, wherein the bonding composition comprises mannitol, maltitol, isomalt, erythritol, sorbitol, xylitol, or a combination thereof.

16. The gummy composition of claim 1, wherein the bonding composition consists essentially of mannitol, maltitol, isomalt, or a combination thereof.

17. The gummy composition of claim 1, wherein the composition is substantially free of a sugar having a glycemic index of more than 20, a sugar alcohol, or any sugar substitute.

18. The gummy composition of claim 1, wherein the gummy composition comprises the gum composition from about 20% to about 60% w/w.

19. The gummy composition of claim 1, wherein the gummy composition comprises the gelling composition from about 0.5% to about 20% w/w.

20. The gummy composition of claim 1, wherein the gummy composition comprises the carbohydrate-based gelling agent from about 0.5% to about 5% w/w.

21. The gummy composition of claim 1, wherein the gummy composition has a pH of at least about 6.

22. The gummy composition of claim 1, further comprising a bioactive composition, wherein the bioactive composition comprises an herbal composition, an active pharmaceutical agent (API), an antioxidant composition, a vitamin composition, a mineral composition, an amino acid composition, a probiotics composition, a prebiotics composition, or a combination thereof.

23. The gummy composition of claim 1, wherein the herbal composition comprises notoginseng, ginger, bornel, Ajuga nipponensis Makino, Chinese yam, Honeysuckle, chrysanthemum, chamomile, lmperata cylindrinca, or a combination thereof.

24. The gummy composition of claim 1, wherein the API comprises an antihistamine, an antitussive, a decongestant, an expectorants, an anti-cold or flu agent, an analgesics, an anti-inflammatory, a sleep aid, an anti-heartburn agent, an anti-gas agent, an anti-GERD agent, an anti-diarrhea agent, a laxative, an anti-smoking agent, a motion sickness medication, or a combination thereof.

25. The gummy composition of claim 1, wherein the API comprises loratadine, diphenhydramine, cetirizine, fexofenadine, chlorpheniramine, ibuprofen, aspirin, naproxen, acetaminophen, guaifenesin, dextromethorphan, aminophylline, diphenhydramine, ranitidine, famotidine, omeprazole, simethicone, pyrantel pamoate, caffeine, nicotine, or a combination thereof.
